(19) United States
(12) Patent Application Publication
(10) Pub. No.: US 2026/0272684 A1
(43) Pub. Date: Sep. 17, 2026

(54) STENT FOR LUMINAL TISSUES

(71) Applicant: MERIL LIFE SCIENCES PVT LTD, Vapi (IN)

(72) Inventors: PRAMOD KUMAR MINOCHA, Vapi (IN); DEVESHKUMAR MAHENDRALAL KOTHWALA, Surat (IN); ARPIT PRADIPKUMAR DAVE, Silvassa (IN); MAHESH LAXMAN PAWAR, Nashik (IN)

(73) Assignee: MERIL HEALTHCARE PVT LTD, Vapi, GJ (IN)

(21) Appl. No.: 19/470,463

(22) PCT Filed: Feb. 16, 2024
(86) PCT No.: PCT/IN2024/050165

§ 371 Date: Sep. 29, 2025

(30) Foreign Application Priority Data
Mar. 30, 2023 (IN) 202323024048

(51) Int. Cl. A61F 2/915

(52) U.S. Cl. CPC A61F 2/915; A61F 2002/91575

(57) ABSTRACT

The present invention discloses a stent including at least one first row of first struts and a plurality of second rows of second struts. The first struts extending circumferentially and disposed both at a proximal end and a distal end. The second struts extending circumferentially and axially disposed between the first rows. Further, the stent includes at least one first row of cells, at least one second row of cells, a plurality of third row of cells and a plurality of fourth row of cells. The at least one first row of cells disposed each at the proximal end and the distal end. The at least one second row of cells disposed adjacent to each of the first row of cells. The plurality of third row of cells and the plurality of fourth row of cells are placed between the second row of cells.

### FIELD OF INVENTION

The present invention relates to self-expanding stent. The invention particularly relates to a self-expanding venous stent with high radial strength and flexibility required for low thickness vein vasculature/anatomy (compared to artery).

### BACKGROUND OF THE INVENTION

The blood vessel walls like arteries and veins are made up of tissues that may get damaged due to various causes leading to constriction and perforation of the vessels causing clinically relevant diseases like vessel perforation, ischemia, aneurysms, etc. To maintain patency/structure of blood vessels, stenting in veins are performed such as inferior vena cava, iliac veins, femoral veins, and popliteal vein. Stents are generally used to keep the vessel lumen intact, open and to facilitate normal blood flow. Stent may be made up of stainless steel, cobalt chromium alloy, nitinol alloy, etc. Stent made up of nitinol alloy is widely used for making self-expanding stents, especially for peripheral vascular applications which include stenting in iliac, femoral popliteal and infra-popliteal arteries and veins. Stents implanted in the peripheral arteries and veins are exposed to high mechanical stresses from the surrounding environment, for example bending of the knee, twisting and compression during walking or running. Nitinol stents are able to handle these external forces better than other shape memory materials due to its characteristic properties of super elasticity, extreme stress and kink resistance. Nitinol stents are well suited for the tortuous vessel pathways of peripheral arteries and veins.

Based on the mechanical attributes, anatomy and the physiology of the vessel, the corresponding stent has to meet requirements for safe performance. The biological and mechanical response of a vessel to the stent is another fact that should be considered for long-term performance of the stent. Venous stents are designed for venous disease treatment, and in comparison to arterial stents, the venous stent has not been sufficiently investigated. The mechanical properties of venous stents are not necessarily same as arterial stent, thus, it is not always effective to treat a venous disease by arterial stent. The requirement of venous vasculature comprises of high radial force, crush resistance and excellent flexibility to sustain in the venous environment.

Conventionally, nitinol stent for venous vasculature exhibits greater wall thickness having desired strength and flexibility to treat venous vasculature. Due to greater wall thickness of stent, it requires longer duration for endothelial layer formation and thus relatively more time to heal at treatment site. Stent comprises only longer straight link may damage blood vessel during bending movement of the stent as crowns go outward and thus it may affect flexibility of the stent post implantation. The closed cells are connected with links which are located at the centre of the closed cells located at the proximal and distal end thereby hampering the flexibility and flaring capabilities of the stent.

Therefore, there is a need for development of a stent which can overcome the drawbacks of the existing stents.

### SUMMARY OF THE INVENTION

The present invention relates to a stent to be deployed within a luminal tissue. The stent includes a proximal end, a distal end, at least one first row of first struts and a plurality of second rows of second struts. The first row of first struts extending circumferentially and disposed both at the proximal end and the distal end. Every two consecutive first struts defining at least one of a plurality of peaks or a plurality of troughs. The plurality of second rows of second struts extending circumferentially and axially disposed between the first rows of first struts. Every two consecutive second struts defining at least one of a plurality of peaks or a plurality of troughs. Further, the stent includes a plurality of rows of cells formed by coupling one first row of first struts with one adjacently disposed second row of second struts or two adjacently disposed second rows of second struts. The plurality of rows of cells including at least one first row of cells, at least one second row of cells, a plurality of third row of cells and a plurality of fourth row of cells. The at least one first row of cells disposed each at the proximal end and the distal end. The first row of cells including a plurality of closed cells. The at least one second row of cells disposed adjacent to each of the first row of cells. The second row of cells including a plurality of partially open cells. The plurality of third row of cells alternates with the plurality of fourth row of cells. The plurality of third row of cells and the plurality of fourth row of cells are placed between the second row of cells. The third row of cells including a plurality of partially open cells. The fourth row of cells including a plurality of open cells. A number of partially open cells in each of the second and third row of cells is more than a number of open cells in each of the fourth row of cells but less than a number of closed cells in each of the first row of cells.

The foregoing features and other features as well as the advantages of the invention will become more apparent from the following detailed description, which proceeds with reference to the accompanying figures.

### BRIEF DESCRIPTION OF THE ACCOMPANYING DRAWINGS

The summary above, as well as the following detailed description of illustrative embodiments, is better understood when read in conjunction with the apportioned drawings. For the purpose of illustrating the present disclosure, exemplary constructions of the disclosure are shown in the drawings. However, the disclosure is not limited to specific methods and instrumentalities disclosed herein. Moreover, those in the art will understand that the drawings are not to scale.

FIG. 1 shows the stent 1 according to an embodiment of the present invention.

FIG. 1A shows the various dimension areas of the stent 1 according to an embodiment of the present invention.

FIG. 2 shows the various sections of the zig zag struts of the stent 1 according to an embodiment of the present invention.

FIG. 2A shows the various dimension areas of the zig zag struts of the stent 1 according to an embodiment of the present invention.

FIG. 3 shows closed cell units of the stent 1 according to an embodiment of the present invention.

FIG. 4 shows the stent 1 according to an embodiment of the present invention in the contacted state within the catheter.

FIG. 5 shows the stent 1 according to an embodiment of the present invention in the partially deployed state in combination with the catheter.

FIG. 6 shows the stent 1 according to an embodiment of the present invention in the fully deployed state in combination with the catheter.

FIG. 7 shows a stent 100 according to an embodiment of the present invention.

FIG. 7a shows the stent 100 in a contracted state according to an embodiment of the present invention.

FIG. 8 shows the stent 1 and the stent 100 according to an embodiment of the present invention in the fully deployed state.

FIG. 9a shows the stent 100 according to an embodiment of the present invention in the contacted state within the catheter.

FIG. 9b shows the stent 100 according to an embodiment of the present invention in the partially deployed state in combination with the catheter.

FIG. 9c shows the stent 100 according to an embodiment of the present invention in the fully deployed state in combination with the catheter.

### DETAILED DESCRIPTION OF THE INVENTION

Prior to describing the invention in detail, definitions of certain words or phrases used throughout this patent document will be defined: the terms “include” and “comprise”, as well as derivatives thereof, mean inclusion without limitation; the term “or” is inclusive, meaning and/or; the phrases “coupled with” and “associated therewith”, as well as derivatives thereof, may mean to include, be included within, interconnect with, contain, be contained within, connect to or with, couple to or with, be communicable with, cooperate with, interleave, juxtapose, be proximate to, be bound to or with, have a property of, or the like; Definitions of certain words and phrases are provided throughout this patent document, and those of ordinary skill in the art will understand that such definitions apply in many, if not most, instances to prior as well as future uses of such defined words and phrases.

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Although the operations of exemplary embodiments of the disclosed method may be described in a particular, sequential order for convenient presentation, it should be understood that the disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed herein. Moreover, for the sake of simplicity, the attached figures may not show the various ways in which the disclosed system, method, and apparatus can be used in combination with other systems, methods, and apparatuses.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments. These features and advantages of the embodiments will become more fully apparent from the following description and apportioned claims, or may be learned by the practice of embodiments as set forth hereinafter.

In order to achieve the desired objectives and to overcome the drawback of the prior art, the inventors have come up with a flexible stent for deployment at the blocked blood vessel specially in venous system, with desired thinner strut width without compromising the other desired characters of the stent such as radial strength, flexibility, ease of deployment, faster growth of tissue around the stent for patient stabilization etc.

The stent of the present invention is a hybrid design having open cells and closed cells geometry. The hybrid design consists of closed cells at the proximal and distal end connected with each other by short straight links and consecutive axially positioned open cells which are interlinked with each other as well as the closed cells by long straight link. Long straight link is interlinked with the cells in such a way that it helps to enhance flexibility of the stent without compromising structural integrity.

Open cells are distributed unequally which helps to achieve desired flexibility during bending without compromising structural integrity and to maintain smooth patency of the stent. This also prevent blood vessel from damage post implantation of stent.

The stent is formed by zigzag shape of strut having multiple inter radiuses which reduce the stress and helps to achieve good radial strength. Further, different strut width at different portions of the stent helps to achieve desired radial strength by distributing stress equally at every portion of the stent body.

To enhance the performance of the stent, optimal strut width and reduced strut wall thickness of the stent help to achieve the desired radial strength to maintain patency in the blood vessel.

Closed cell design forms rings or crowns which are interlinked with the help of short straight link positioned at offset from the center of the cells. Due to offset link positioned at both ends of stent, the desired angle to form a flare shape is possible which helps the stent to contact more surface area of the blood vessel thereby improving the holding capacity thus preventing migration of the stent. This also avoids edge injury during deployment and post implantation.

The stent geometry helps to smoothly and easily load into the catheter without overlapping of strut and provides uniform crimping profile and thus helps in uniform self-expansion of stent without affecting structural integrity of the stent.

The embodiments of the invention are now more fully described with the reference to the drawings.

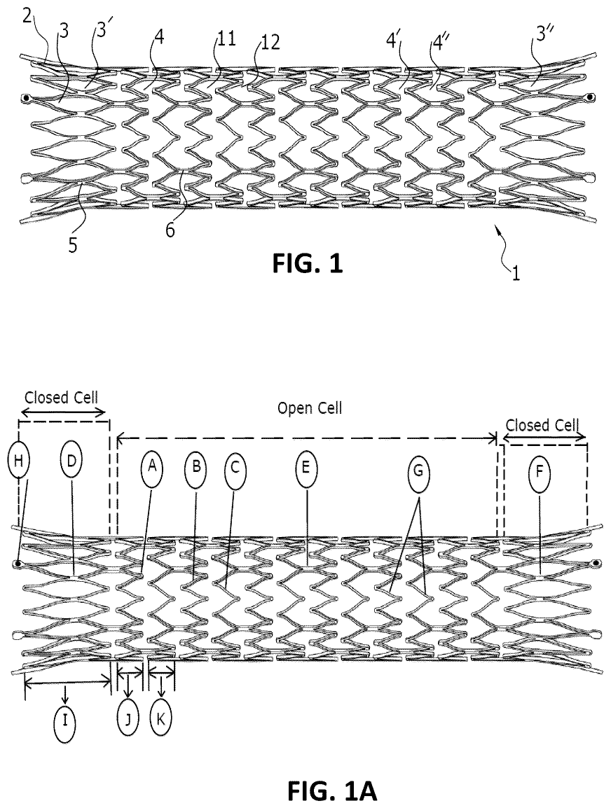

FIG. 1 shows a low thickness flexible tubular stent (1) for blood vessel, specially venous obstruction removal application according to a preferred embodiment of the present invention.

For the sake of clarity and uniformity, as shown in FIG. 1, the left side of the stent is termed as the proximal end while the right hand side of the stent is the distal end.

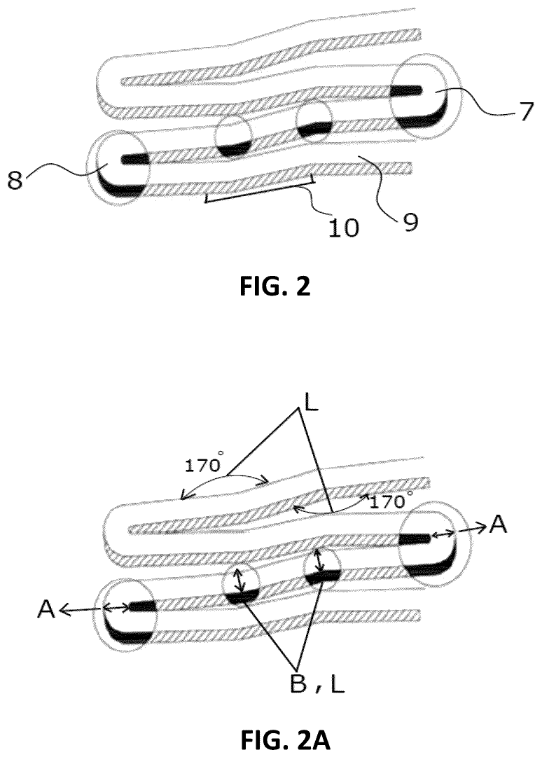

The stent (1) is made of struts (2) of suitable material such as nitinol and are formed continuously in a zigzag pattern extending in a circumferential direction to form plurality of ring-shaped stent cell units or crowns (3′, 3″, 4′, 4″). As can be seen in FIG. 2 and as is known to a skilled person that the continuous zig zag pattern of the strut consists of sharp bend portions forming peak and trough (7, 8) and an arm section (9) extended between each said peak and trough.

The ring shaped stent cell units are placed adjacent to each other in the axial direction of the stent body from a proximal to distal end to form a tubular stent structure. The outer circumference of the said stent units is expandable in the radial direction from a contracted state to an expanded state.

Two of the said axially adjacent ring-shaped cell stent units (3′, 3″) are connected by short links (5) suitably to form ring shaped closed cell stent units (3). The ring shaped closed cell stent units (3) are formed at both the proximal and distal end of the stent body.

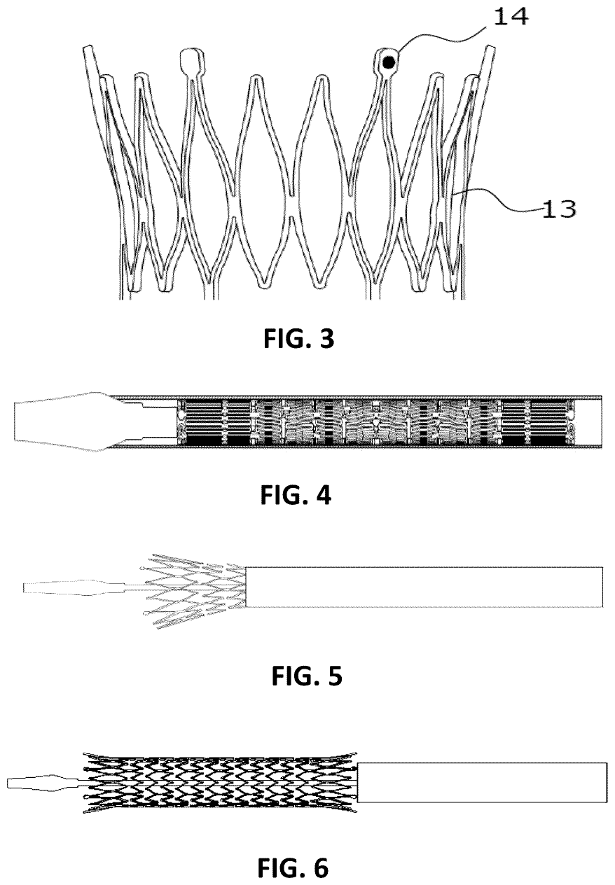

As shown in FIG. 3, the said closed cell stent units (3′, 3″) are formed by connecting each adjacent peak and trough of the two axially adjacent zigzag struts through short links (5).

In an embodiment, as shown in FIG. 3, the closed cells are interlinked with the help of short straight link (5) positioned slightly offset (13) from the center of the closed cells to achieve the desired angle of flare shape ranging from 165° to 175° at both the ends for desired holding capacity of the stent. Such an angle of flare also engages with greater contact surface area of the blood vessel thus preventing migration of stent from the treatment site.

In an embodiment, the closed cell stent units have 18 number of closed cells both at the proximal and distal end. However, the cell number may vary according to the diameter of the stent.

Diameter of the expanded stent ranges from 8 mm to 25 mm, more preferably from 10 mm to 22 mm. The expanded stent length ranges from 30 mm to 200 mm, more preferably 40 mm to 160 mm.

In an embodiment, the struts (2) of the closed cell units have uniform strut width.

The other said ring shaped stent cell units (4′, 4″) are interconnected with each other and also to the said closed cell stent units (3) at the proximal and distal end of the stent through long straight links (6) in the stent axial direction in a manner suitable to form plurality of open cell stent units (4), thereby positioning the said plurality of interconnected open cell stent units (4′, 4″) axially along the length of the stent body between the said closed cell stent units (3′, 3″) at the proximal and distal end of the stent body.

In one aspect of the invention, the said open cell stent units are formed by connecting a few of the selected adjacent peaks and trough of the two axially adjacent zigzag struts through long straight links (6), whereby each open cell units (4) consists of 18 or 15 i.e. unequal number of open cells (11, 12).

The open cell stent units with unequal number of cells are axially positioned in a consecutive manner along the length of the stent so that if one open cell unit (4′) consists of 18 cells (11), the next axially placed open cell unit (4″) consists of 15 cells (12). However, as would be clear to a skilled person, the number of units of open cells is not constant and may vary according to the varying circumference of the stent. For example, open cells may be distributed in unequal numbers ranging from 10 to 25 in the open cells depending on the stent diameter.

According to the invention, the zigzag patterned strut of the open cell stent units (4′, 4″) has different strut widths in the peak/trough (7, 8) section and the arm section (9), wherein the zigzag struts of the open cell stent units have a greater strut width in the peak/trough section than the arm section.

According to the invention, as shown in FIG. 2, the arm section (9) of the zigzag strut has a section (10) with at least two opposite curvatures along its length. In an aspect, as shown in FIG. 2A, the said two opposite curvatures in the arm section has an angle (L) of around 170 degree. However, number of curvatures and angle may vary according to the size of the stent and the required strut length for achieving desired radial strength.

FIGS. 1A, 2A and the following Table 1 provide various preferred dimensions of the various parts of the stent of the preferred embodiment of the present invention. The dimensions are applicable for a stent dia of 14, 16 mm and stent length of 40, 60, 80, 100, 120, 160 mm.

TABLE 1

| Refer | | | Post Processing |
|---|---|---|---|
| FIGS. | | Post Processing | Range of |
| 1A & | | Broader range | Preferred |
| 2A | Stent design description | of Dimension | Dimension |
| A. | Strut Width Peak and | 240 μm-320 μm | 250 μm-300 μm |
| | Trough sections | | |
| B. | Strut Width (18 cell | 150 μm-200 μm | 160 μm-190 μm |
| | Open/Closed cell strut | | |
| | width) | | |
| C. | Strut Width (15 cell open | 150 μm-200 μm | 160 μm-190 μm |
| | cell strut width) | | |
| D. | Short Straight Link | 230 μm-280 μm | 240 μm-270 μm |
| | (Proximal) width | | |
| E. | Long Straight Link width | 400 μm-470 μm | 410 μm-460 μm |
| F. | Short Straight Link width | 230 μm-280 μm | 240 μm-270 μm |
| | (Distal) | | |
| G. | Wall Thickness | 150 μm-400 μm | 300 μm-380 μm |
| H. | Marker Cavity Dia | 450 μm-580 μm | 470 μm-560 μm |
| I. | Closed Cell Length | — | 8 mm-12 mm |
| | (Single Crown 18 cell) | | |
| J. | Open Cell Length (Single | — | 3 mm-6 mm |
| | Crown 18 cell) | | |
| K. | Open Cell Length (Single | — | 3 mm-6 mm |
| | Crown 15 cell) | | |
| L. | Both Opposite Curvature | — | 170° |
| | Angle | | |
| M. | Flared shape Angle on | — | 165°-175° |
| | Deployment | | |

In an aspect, the peak/trough section has a strut width 240 μm-320 μm, after electropolishing. More preferably, the range can vary between 250 μm-300 μm.

In an aspect, the strut width of the arm section (9) of the open cell stent units consisting of 18 cells (11) is 150 μm-200 μm and more preferably from 160 μm-190 μm and that of the arm section (9) of open cell stent units consisting of 15 cells (12) is 150 μm-200 μm and more preferably ranges from 170 μm-190 μm after electropolishing.

In a preferred embodiment, the width of the said short link (5) is 230 μm-280 μm and more preferably the range can vary from 240 μm-270 μm. The width of the said long link is in the range between 400 μm and 470 μm and more preferably between 410 μm and 460 μm, after electropolishing.

Therefore, in one embodiment, in respect of the whole stent the proximal and distal end located closed cell short straight link width is in the range from 230 μm-280 μm and more preferably from 240 μm to 270 μm, while the intermediate open cell long straight link width is in the range from 400 μm-470 μm and preferably from 410 μm to 460 μm.

This different link widths help to achieve desired flexibility of the stent and provide bending radius ranging from 30 mm to 40 mm for 100 mm length stent, for maintaining smooth patency of stent and prevent damage of blood vessel during bending movement of stent.

In an embodiment, said closed cell length (3, 3″) is in the range between 8 mm and 12 mm.

In an embodiment, the said open cell length in 18 open cell containing unit ranges from 3 mm to 6 mm and the said open cell length in 15 open cell containing unit ranges from 3 mm to 6 mm.

In an embodiment the wall thickness of the strut preferably ranges from 300 to 380 μm which is thinner than the presently available stent for venous obstruction removal application.

In one aspect, different wall thickness of the strut may be used in different areas of the stent.

In one embodiment stent has two variable wall thickness in which 60 mm stent has same thickness at proximal and distal end ranging from 380 μm to 420 μm while having lesser thickness at the intermediate region ranging from 350 μm to 400 μm.

In another embodiment the stent may have three variable thicknesses in a 60 mm stent that is distributed in five equal regions of 12 mm in such way that proximal and distal end region have highest wall thickness ranging from 380 μm to 420 μm, the very next region of the proximal and distal end has lesser wall thickness ranging from 350 μm to 400 μm and the central region of the stent has the least thickness ranges from 320 μm to 380 μm.

This feature of the stent adds more flexibility at intermediate region and better holding capacity & strength at both ends of the stent. Thus, combining of strength and flexibility helps to maintain crushing ability due to high forces and enough flexibility to align and accommodate with implantation environment.

In an embodiment, the desired expanded diameter of the stent ranges from 8 mm to 25 mm, more preferably from 10 mm to 22 mm. The expanded stent length ranges from 30 mm to 200 mm, more preferably 40 mm to 160 mm.

Expanded stent has reduced wall thickness ranges from 150 μm to 400 μm, more preferably from 300 μm to 380 μm. Reduced wall thickness of stent beneficial towards tissue growth rate on stent over period of time.

In a preferred embodiment, close cells are formed in a diamond shape as shown in FIG. 1 which helps in equal expansion of stent.

In an aspect, both ends of stent have arrangements to attach number of radiopaque markers (14) ranging from 2 to 15, more preferably from 4 to 10 which help to track and position the stent at treatment site under fluoroscopic process.

The marker hole diameter ranges from 450-580 μm, more preferably from 470-560 μm.

The process for manufacturing the stent comprise several processes such as laser cutting, grinding, honing, shape setting, sand-blasting and electro-polishing processes. To get the desired shape of the stent required design of stent which is cut from tube under laser cutting operation where hybrid design of the stent is formed by laser cutting machine. During this process, burrs are generated as the metal gets melt due to high frequency of the laser beam.

After laser cutting process, grinding process is performed to remove burrs which are generated during laser cutting operation. To perform grinding operation, several abrasive files were used as per the inner diameter of laser cut tube. After grinding process, honing process is performed to get smooth surface from inner diameter of the laser cut tube. This process is performed by applying fluids like glycol and Alconox® detergent powder i.e. a mixture of Sodium tripolyphosphate, Sodium Alkyl benzene Sulfonate and Tetrasodium Pyrophosphate which is anionic detergent used for manual and ultrasonic cleaning and is excellent replacement for corrosive acids and damaging solvents.

In another embodiment, grinding is performed in such a manner to achieve variable strut thickness at different regions of the stent. In order to achieve variable thicknesses at different regions of the stent variable size files ranging from 2.70 mm to 3.75 mm are used to achieve precise bore diameter as required. Masking is done to the desired area of stent followed by electro-polishing process over the surface of stent. The process is repeated until desired wall thickness is achieved.

After the grinding & honing process, desired shape of stent is achieved by performing multiple steps of shape setting process using mandrel or mold in high temperature oven or furnace. Further, open cells are equally adjusted with the help of needle and each strut is also adjusted to obtain desired shape of the stent by using different mandrels or mold. Flare shape of the stent at both ends is obtained with the help of taper ring which is assembled with the mandrel by Allen screw at both side of the stent. To check the length of the stent, gauge is prepared which comprises grooves at both ends where taper ring with the stent is placed to confirm the stent overall length. By assembling of the stent and taper ring over mandrel, shape setting process is performed to get the desired shape of the stent. Stent is dipped into Aluminum white sand at specific temperature for specified time for effective shape setting.

Shape setting process enhances internal grain hardness and relieves stress. On the other hand, inappropriate temperature leads to the destruction of shape memory of stent.

Sand blasting is used to provide highly smooth surface. Devices such as stents, shunts, cages, etc. are commonly blasted to de-burr them and to remove the oxide layers from surfaces and also to remove pulse marks and striations left by laser machining, cut or remove laser slag. This also decreases the propensity for micro cracking and lightly texture the surface to improve adhesion characteristics during sand blasting. Aluminum oxide power is struck on the device surface at certain velocity. Due to abrasion, the micro cracks and burrs are removed. Highly smooth surface with the increase in surface texture of stent after sand blasting is obtained. After sandblasting, electro-polishing is done.

In the present invention, mixture of perchloric acid and acetic acid is used during the process. To achieve electro-polishing of a rough surface, the protruding parts of a surface must dissolve faster than the recesses. By performing electro-polishing process over the stent, smooth surface finish without any cracks is obtained. The oxide layer generated during sandblasting process is also removed. If the sand blasting process is not proper, it directly affects the stent property during electro-polishing process and also affects the stent mechanical properties.

After electro-polishing process, radiopaque markers are attached or integrated into the design of the stent. Tantalum markers of riveted, coined, ellipses, rectangular shape are attached into stent by laser welding process. The presence of laser cut nitinol marker in addition to tantalum marker offer enhancement in radiopacity.

Further, the stent is led towards loading process where it is loaded with the help of quill and crimping machine. By providing relative motion of both the stent and catheter, the stent is smoothly and uniformly loaded onto the delivery catheter ranging from 8F to 12F as shown in FIG. 4 without affecting the stent's structural integrity.

For deployment of the stent, as per the standard practice access site is prepared. Then, guide wire of 0.035″ (0.89 mm) is advanced to the location of lesion. If necessary, the treatment site is pre-dilated. After pre-dilation, the guide wire is kept for stent system advancement. The delivery system is placed over the guide wire and the delivery system is advanced as a single unit through the hemostatic valve of the introducer sheath. The stent is placed at the treatment site by advancement of delivery system till stent reaches the center region of the lesion as confirmed by stent marker position. Once the distal stent radiopaque markers start separating, the stent starts getting partially but uniformly deployed as shown in FIG. 5. It is further led to full deployment as shown in FIG. 6 at the treatment site without affecting structural integrity. The blood vessel which gets narrowed/obstructed and treated with venous stent leads towards reconstruction of the blood vessel by maintaining patency and regulate the blood flow.

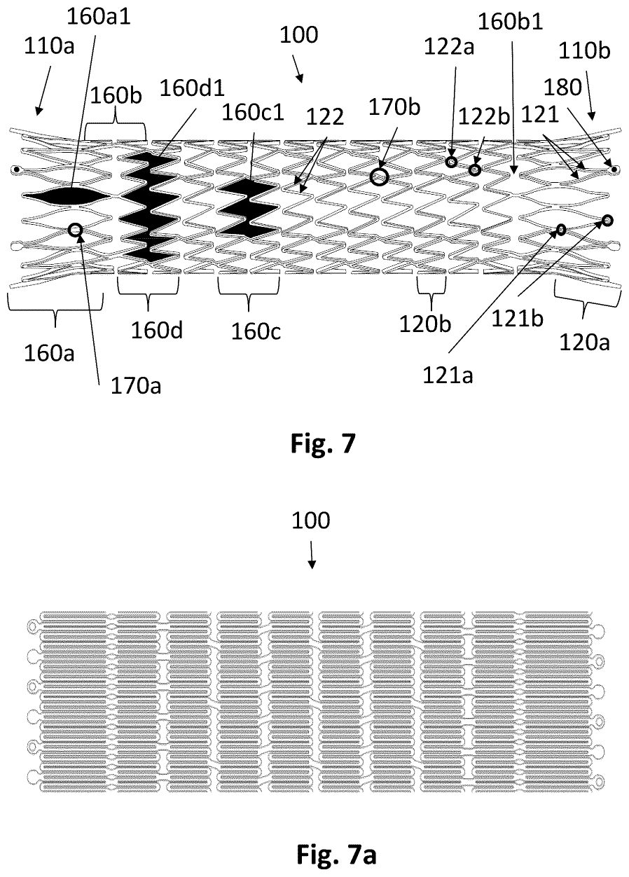

FIG. 7 depicts another exemplary embodiment of a stent 100. In an exemplary embodiment, similar to stent 1, the stent 100 is implanted within a luminal tissue (for example, the venous vasculature) to reduce luminal obstruction or the like. The stent 100 may be made of a shape memory material including but not limited to nitinol, cobalt-chromium, Stainless Steel, etc. In an exemplary embodiment, the stent 100 is made of nitinol. The stent 100 may be a self-expandable implant that is radially compressible from an expanded state (as shown in FIG. 7) to a contracted state (as shown in FIG. 7a) and radially expandable from the contracted state to the expanded state.

FIG. 7 depicts the stent 100 in its expanded state. The stent 100 exhibits excellent radial strength against compressive forces at an implantation site thereby making the stent 100 prolapse resistant. In other words, the stent 100 can easily resist and endure the compressive forces at the implantation site in both during and post-deployment of the stent 100. The radial strength of the stent 100 may depend upon the dimensions of the stent 100. In an exemplary embodiment, radial strength of a 60 mm long stent 100 having a diameter of 16 mm ranges from 50N to 60N. Further, the stent 100 provides increased flexibility and kink resistance.

The stent 100, in its contracted state, may define a uniform crimping profile (or diameter). In an exemplary embodiment, the crimping profile of the stent 100 is less than 10Fr. The stent 100 in its contracted state may be smoothly and easily loaded within a catheter. Once the said catheter is advanced through the vasculature to the implantation site, the stent 100 is allowed to self-expand by sliding the catheter off the stent 100 thus, implanting the stent 100 at the implantation site.

The stent 100 includes a proximal end 110a, a distal end 110b and a plurality of rows of struts extending axially between the proximal end 110a and the distal end 110b.

The plurality of rows of struts may include at least two first rows 120a and a plurality of second rows 120b. The first rows 120a may be disposed at the proximal end 110a and the distal end 110b of the stent 100. In an exemplary embodiment, as shown in FIG. 7, the stent 100 includes one first row 120a at the proximal end 110a and one first row 120a at the distal end 110b.

The second rows 120b are axially disposed between the first rows 120a. The number of second rows 120b may depend upon a length of the stent 100. The length of the stent 100, in the expanded state, may range from 30 mm to 200 mm. In an exemplary embodiment, in the expanded state, the length of the stent 100 is 60 mm.

The first row 120a may include a plurality of first struts 121 extending circumferentially. The number of first struts 121 may depend upon a diameter of the stent 100. The diameter of the stent 100, in the expanded state, ranges from 8 mm to 25 mm. In an exemplary embodiment, in the expanded state, the diameter of the stent 100 is 16 mm.

The plurality of first struts 121 may make a pre-defined pattern. As shown in FIG. 7, the plurality of first struts 121 makes a zig-zag pattern thereby forming a plurality of peaks 121a and a plurality of troughs 121b. The peaks 121a and troughs 121b may have a width ranging from 220 μm to 320 μm. In an exemplary embodiment, the width of the peaks 121a and the troughs 121b is 230 μm.

In the context of the embodiment of the stent 100 as shown in FIG. 7, the peaks 121a are defined as the coupling between two consecutive first struts 121 that converge towards the proximal end 110a. Similarly, the troughs 121b are defined as the coupling between two consecutive first struts 121 that converge towards the distal end 110b.

The first struts 121 may include a length ranging from 5 mm to 15 mm. The first struts 121 may include a width ranging from 230 μm to 320 μm. Further, the first struts 121 may include a wall thickness ranging from 350 μm to 450 μm. In an exemplary embodiment, the length, width and wall thickness of the first strut 121 is 10 mm, 250 μm and 390 μm respectively. The first struts 121 may have a predefined shape including but not limited to straight, curved, circular, etc. In an exemplary embodiment, as shown in FIG. 7, the first strut 121 is straight shaped.

Similar to the first row 120a, the second row 120b may include a plurality of second struts 122 extending circumferentially. The number of second struts 122 may depend upon the diameter of the stent 100. The number of first struts 121 in the first row 120a and the number of second struts 122 in the second row 120b may be equal across the length of the stent 100.

Similar to the first struts 121, as shown in FIG. 7, the plurality of second struts 122 makes a zig-zag pattern thereby forming a plurality of peaks 122a and a plurality of troughs 122b. The peaks 122a and troughs 122b may have a width ranging from 220 μm to 320 μm. In an exemplary embodiment, the width of the peaks 122a and the troughs 122b is 230 μm.

In the context of the embodiment of the stent 100 as shown in FIG. 7, the peaks 122a are defined as the coupling between two consecutive second struts 122 that converge towards the proximal end 110a. Similarly, the troughs 122b are defined as the coupling between two consecutive second struts 122 that converge towards the distal end 110b.

The second strut 122 may have a length shorter than the first struts 121, thus, the first row 120a (located at the proximal end 110a and distal end 110b) is flared outwardly thereby defining a predefined angle with a longitudinal axis (not shown) passing through the stent 100. The predefined angle ranging from 160° to 190°. In an exemplary embodiment, the proximal and distal ends 110a, 110b are flared by 168°. The flared proximal end 110a and distal end 110b helps in proper deployment of the stent 100 at the implantation site. The flared proximal end 110a and distal end 110b helps the stent 100 to have more contact area with respect to the implantation site thereby preventing migration of the stent 100 after implantation and increasing holding capacity of the stent 100. The increase in contact area further helps to reduce the time required to heal after the stent 100 is deployed at the implantation site. This said flared proximal and distal ends 110a, 110b also helps prevent edge injury during deployment and post implantation of the stent 100.

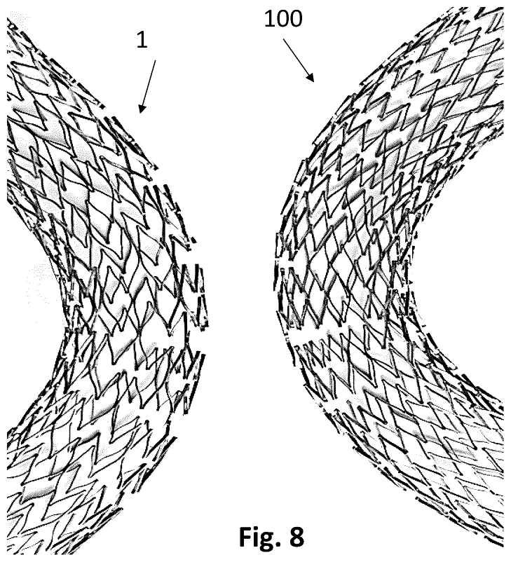

Further, due to the presence of shorter second strut 122, the stent 100 prevents formation of intermittent gaps and protrusions (compared to stent 1 as shown in FIG. 8) that may have a traumatic effect on the surrounding vessel.

The length of the second struts 122 may range from 3 mm to 9 mm. The second strut 122 may have a width ranging from 130 μm to 190 μm. Further, the second struts 122 may include a wall thickness ranging from 350 μm to 450 μm. In an exemplary embodiment, the length, width and wall thickness of the second strut 122 is 3.7 mm, 150 μm and 390 μm respectively. The second struts 122 may have a predefined shape including but not limited to straight, curved, circular, etc. In an exemplary embodiment, as shown in FIG. 7, the second strut 122 is straight shaped. The straight and relatively short second struts 122 minimizes friction with the vessel and makes the stent 100 kink resistant against the curvature of a vessel (implantation site).

The first rows 120a and the second rows 120b may be selectively coupled to form a plurality of rows of cells (described below). In other words, one first row 120a of first struts 121 may be selectively coupled to one adjacently disposed second row (120b) of second struts (122) or two adjacently disposed second rows 120b of second struts 122 may be selectively coupled to each other to form the plurality of rows of cells. The plurality of rows of cells includes at least two first row of cells 160a, at least two second rows of cells 160b, a plurality of third rows of cells 160c and a plurality of fourth row of cells 160d. The first row of cells 160a may have an axial length (or height) ranging from 5 mm to 15 mm. The second, third and fourth row of cells 160b, 160c, 160d may have an axial length (or height) ranging from 3 mm to 15 mm.

In an exemplary embodiment, as shown in FIG. 7, one first row of cells 160a is disposed at the proximal end 110a and one first row of cells 160a is disposed at the distal end 110b. The first rows of cells 160a is formed by coupling the consecutive first struts 121 of the first row 120a to the consecutive second struts 122 of an adjacent second row 120b via a plurality of first links (or short straight links) 170a thereby making a plurality of closed cells (or crowns) 160a1. Each closed cell 160a1 is defined by two consecutive first struts 121 of a first row 120a, two consecutive second struts 122 of a second row 120b and two first links 170a (described below).

Each first row of cells 160a may include a predefined number of closed cells 160a1. The predefined number of closed cells 160a1 may range from 15 to 20 closed cells 160a1 depending upon the diameter of the stent 100. In an exemplary embodiment, as shown in FIG. 7, the stent 100 includes 18 closed cells 160a1 in each of the first rows of cells 160a.

At the proximal end 110a, the troughs 121b of the first struts 121 may align with the peaks 122a of the second struts 122. Each closed cell 160a1 is made by coupling each trough 121b of the first struts 121 of one first row 120a to respective peak 122a of the second struts 122 of adjacent second row 120b with the help of first links 170a thereby defining a closed area therein.

Conversely, at the distal end 110b, the peaks 121a of the first struts 121 may align with the troughs 122b of the second struts 122. Each closed cell 160a1 is made by coupling each peak 121a of the first struts 121 of one first row 120a to respective trough 122b of the second struts 122 of adjacent second row 120b with the help of first links 170a.

The plurality of first links 170a may have a pre-defined shape including but not limited to straight shaped, S-shaped, Zig-Zag, etc. In an exemplary embodiment, as shown in FIG. 7, the shape of the first links 170a is straight. The first links 170a may have a length ranging from 1 mm to 3 mm. The first links 170a may have a width ranging from 230 μm to 300 μm. In an exemplary embodiment, the length and width of the first links 170a is 1.1 mm and 230 μm respectively.

As shown in FIG. 7, at least one second rows of cells 160b may be disposed adjacent to each of the first row of cells 160a. Alternatively, not shown, at least one third row of cells 160c or one fourth row of cells 160d may be disposed adjacent to the first row of cells 160a.

In an exemplary embodiment, as shown in FIG. 7, one second row of cells 160b is disposed towards the proximal end 110a and one second row of cells 160b is disposed towards the distal end 110b, adjacent to the first row of cells 160a. The second rows of cells 160b, adjacent to the first row of cells 160a, are formed by coupling respective peaks 122a or troughs 122b of consecutive second struts 122 of two adjacent second rows 120b via the plurality of first links 170a thereby making a plurality of partially open cells 160b1 (described below).

Each second row of cells 160b may include a predefined number of partially open cells 160b1. The predefined number of partially open cells 160b1 may vary from 5 to 7 partially open cells 160b1 depending upon the diameter of the stent 100. In an exemplary embodiment, as shown in FIG. 7, the stent 100 includes 6 partially open cells 160b1 in each of the second rows of cells 160b.

In an exemplary embodiment, each partially open cell 160b1, adjacent to the first row of cells 160a, is defined by twelve second struts 122 and two first links 170a. In other words, six consecutive second struts 122 of a second row 120b is coupled to six consecutive second struts 122 of an adjacent second row 120b by two first links 170a thereby defining a partially open area therein.

As shown in FIG. 7, the third rows of cells 160c may be alternately disposed with the fourth rows of cells 160d between the second row of cells 160b.

The third row of cells 160c may be structurally similar to the second row of cells 160b except that the third row of cells 160c include a plurality of second links (or long straight links) 170b instead of the first links 170a. In an exemplary embodiment, each partially open cell 160c1 of the third row of cells 160c is defined by twelve second struts 122 and two second links 170b. In other words, six consecutive second struts 122 of a second row 120b is coupled to six consecutive second struts 122 of an adjacent second row 120b by two second links 170b thereby defining a partially open area therein.

The fourth rows of cells 160d are formed by coupling the respective peaks 122a or troughs 122b of consecutive second struts 122 of two adjacent second row 120b via the plurality of second links 170b thereby making a plurality of open cells (or open cell stent units) 160d1 (described below).

Each fourth row of cells 160d may include a predefined number of open cells 160d1. The predefined number of open cells 160d1 may vary from 2 to 4 open cells 160d1 depending upon the diameter of the stent 100. The predefined number of open cells 160d1 may stay the same across the length of the stent 100 or may vary across the length of the stent 100. In an exemplary embodiment, as shown in FIG. 7, the stent 100 includes 3 open cells 160d1 in each of the fourth rows of cells 160d. Thus, the number partially open cells 160b1, 160c1 in each of the second and third row of cells 160b, 160c is more than the number of open cells 160d1 in each of the fourth row of cells 160d but less than the number of closed cells 160a1 in each of the first row of cells 160a. The said relation between the closed cells 160a1, partially open cells 160b1, 160c1 and open cells 160d1 provides excellent flexibility, trackability and torquability to the stent 100.

Each open cell 160d1 is defined by twenty-four second struts 122 and two second links 170b. In other words, twelve consecutive second struts 122 of a second row 120b is coupled to twelve consecutive second struts 122 of an adjacent second row 120b by two second links 170b thereby defining an open area therein.

The plurality of second links 170b may have a pre-defined shape including but not limited to straight, S-shaped, staggered shaped, helically shaped, zig-zag, slant-shaped, etc. In an exemplary embodiment, as shown in FIG. 7, the shape of the second links 170b is slant-shaped. The second links 170b may have a length ranging from 1 mm to 3 mm. The length of the second links 170b may be longer than the first links 170a. The second links 170b may have a width ranging from 320 μm to 470 μm. In an exemplary embodiment, the length and width of the second links 170b is 1.3 mm and 290 μm respectively.

The second links 170b of two adjacent third rows of cells 160c may align with each other or may be disposed at a pre-defined offset with each other. Similarly, the second links 170b of two adjacent fourth rows of cells 160d may align with each other or may be disposed at a pre-defined offset with each other.

In an exemplary embodiment, as shown in FIG. 7, two adjacent third rows of cells 160c include equal number of the second links 170b which are aligned with each other. Further, the second links 170b of two adjacent fourth rows of cells 160d are also equal in number and align with each other. The second links 170b of the third row of cells 160c and the fourth row of cells 160d are circumferentially offset from each other. The said arrangement of the second links 170b of the stent 100 provides excellent kink resistance and flexibility to the stent 100.

Compared to stent 1, the above-described geometry of stent 100 formed by the first struts 121 and the second struts 122 renders the stent 100 to have smooth morphology (as shown in FIG. 8) across the length of the stent 100. In other words, the stent 100 includes an atraumatic smooth surface (i.e., inhibits or reduces strut protrusion) that prevents any injury to the implantation site at the time of deployment and post implantation of the stent 100. The stent 100 retains its atraumatic property even when the implantation site includes torturous morphology.

The stent 100 may be provided with one or more radiopaque markers 180. As shown in the FIG. 7, at least one of the peaks 121a at the proximal end 110a and at least one of the troughs 121b at the distal end 110b is provided with one or more radiopaque markers 180 each.

In an exemplary embodiment, the radiopaque marker 180 may be disposed within a cavity (not shown). The cavity may have a diameter ranging from 450 μm to 580 μm.

Additionally or optionally, the radiopaque markers 180 may be disposed between the proximal end 110a and the distal end 110b of the stent 100.

The stent 100 may be manufactured by following a similar process as that of the stent 1 (described above).

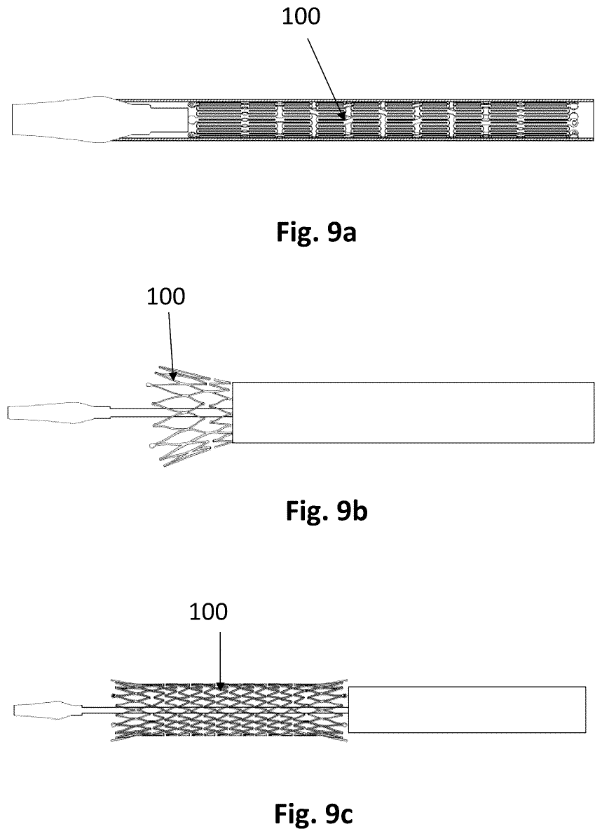

Similarly, the stent 100 is loaded and deployed similar to the stent 1 (described above). FIG. 9a depicts the stent 100 smoothly and uniformly loaded onto the delivery catheter in its contracted state. Further, FIG. 9b depicts the stent 100 being uniformly expanded into a partially deployed state. And, FIG. 9c depicts the stent 100 being completely expanded to its expanded state.

The invention will now be explained with the help of following example.

### Example 1 (Present Invention)

The stent 100 was manufactured by laser cutting a nitinol tube with zigzag strut design according to the present invention. The resultant stent 100 was 60 mm in length and capable of being expanded to 16 mm in diameter. The struts of the stent 100 was straight shaped and had a uniform wall thickness of around 390 μm across the length of the stent 100.

The radial strength of stent 100 was found to be more than 40N.

In response to a compression force exerted on the stent 100, the zigzag pattern of the struts distributed the stress uniformly across the length of the stent 100. Moreover, due to relatively shorter first link 170a (compared to the second link 170b) and presence of shorter first and second struts 121, 122, the stent 100 was bent at a radius of around 20-22 mm without any undesirable surface protrusion and kink (as shown in FIG. 8).

Further, due to relatively longer first struts 121 (compared to the second struts 122), the first row 120a of the stent 100 flared by 168°. The said flaring of the stent 100 prevented migration of the stent 100 from the treatment site. Moreover, the stent 100 had more contact surface area post deployment at the proximal and distal ends 110a, 110b thereby improving the holding capacity of the stent 100.

Due to reduced wall thickness of the struts, the stent 100 had uniform crimping profile which was smoothly loaded into a 10F catheter of a delivery system. Similarly, the stent 100 was uniformly deployed without affecting the structural integrity of the stent 100.

While the invention has been described with the help of preferred embodiments, several modification and improvements are possible without departing from the scope of the invention as described above and as defined in the claims herein below.

The scope of the invention is only limited by the appended patent claims. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

We claim:

1. A stent (100) to be deployed within a luminal tissue, the stent (100) comprising:
a proximal end (110a) and a distal end (110b);
at least one first row (120a) of first struts (121) extending circumferentially and disposed both at the proximal end (110a) and the distal end (110b), every two consecutive first struts (121) defining at least one of a plurality of peaks (121a) or a plurality of troughs (121b);
a plurality of second rows (120b) of second struts (122) extending circumferentially and axially disposed between the first rows (120a) of first struts (121), every two consecutive second struts (122) defining at least one of a plurality of peaks (122a) or a plurality of troughs (122b);
a plurality of rows of cells formed by coupling one first row (120a) of first struts (121) with one adjacently disposed second row (120b) of second struts (122) or two adjacently disposed second rows (120b) of second struts (122), the plurality of rows of cells including:
at least one first row of cells (160a) disposed each at the proximal end (110a) and the distal end (110b), the first row of cells (160a) including a plurality of closed cells (160a1);
at least one second row of cells (160b) disposed adjacent to each of the first row of cells (160a), the second row of cells (160b) including a plurality of partially open cells (160b1);
a plurality of third row of cells (160c) that alternates with a plurality of fourth row of cells (160d), the plurality of third row of cells (160c) and the plurality of fourth row of cells (160d) placed between the second row of cells (160b), the third row of cells (160c) including a plurality of partially open cells (160c1), the fourth row of cells (160d) including a plurality of open cells (160d1);
wherein, a number of partially open cells (160b1, 160c1) in each of the second and third row of cells (160b, 160c) is more than a number of open cells (160d1) in each of the fourth row of cells (160d) but less than a number of closed cells (160a1) in each of the first row of cells (160a).

2. The stent (100) as claimed in claim 1, wherein the first row of cells (160a) is formed by selectively coupling peaks (121a) or troughs (121b) of one first row (120a) of consecutive first struts (121) with troughs (122b) or peaks (122a) respectively of an adjacent second row (120b) of consecutive second struts (122) using a plurality of first links (170a).

3. The stent (100) as claimed in any of the above claims, wherein the second row of cells (160b) is formed by selectively coupling peaks (122a) or troughs (122b) of one second row (120b) of consecutive second struts (122) with troughs (122b) or peaks (122a) respectively of adjacent second row (120b) of consecutive second struts (122) using a plurality of first links (170a).

4. The stent (100) as claimed in any of the above claims, wherein the third row of cells (160c) and fourth row of cells (160d) is formed by selectively coupling peaks (122a) or troughs (122b) of one second row (120b) of consecutive second struts (122) with troughs (122b) or peaks (122a) respectively of adjacent second row (120b) of consecutive second struts (122) using a plurality of second links (170b).

5. The stent (100) as claimed in claim 1, wherein a number of first struts (121) in the each of the first row (120a) is equal to a number of second struts (122) in each of the second row (120b) throughout a length of the stent (100).

6. The stent (100) as claimed in claim 1, wherein the first struts (121) and the second struts (122) are straight shaped.

7. The stent (100) as claimed in claim 1, wherein a length of the second struts (122) is shorter than a length of the first struts (121).

8. The stent (100) as claimed in claim 1, wherein the first row (120a) of first struts (121) makes a pre-defined angle with a longitudinal axis, the predefined angle ranging from 160° to 190°.

9. The stent (100) as claimed in claim 1, wherein each of the closed cells (160a1) is defined by two consecutive first struts (121) of one first row (120a), two consecutive second struts (122) of one adjacent second row (120b) and two first links (170a).

10. The stent (100) as claimed in claim 1, wherein each of the partially open cells (160b1, 160c1) is defined by six consecutive second struts (122) of one second row (120b), six consecutive second struts (122) of another adjacent second row (120b) and two first or second links (170a, 170b).

11. The stent (100) as claimed in claim 1, wherein each of the open cells (160c1) is defined by twelve consecutive second struts (122) of one second row (120b), twelve consecutive second struts (122) of another adjacent second row (120b) and two second links (170b).

12. The stent (100) as claimed in claim 10, wherein a length of the second links (170b) is longer than a length of the first links (170a).

13. The stent (100) as claimed in claim 10, the first links (170a) are straight shaped and the second links (170b) are slant-shaped.

14. The stent (100) as claimed in claim 10, wherein a plurality of second links (170b) of two adjacent third row of cells (160c) or two adjacent fourth row of cells (160d) are aligned with each other.

15. The stent (100) as claimed in claim 10, wherein a plurality of second links (170b) of the third row of cells (160c) and the fourth row of cells (160d) are circumferentially offset from each other.
